(19) United States
(12) Patent Application Publication
(10) Pub. No.: US 2002/0098552 A1
(43) Pub. Date: Jul. 25, 2002

(54) Method for producing xanthosine-5'-monophosphate by fermentation using mutant strains of Coryneform bacteria

(75) Inventors: Vitaliy Arkadyevich Livshits, Moscow (RU); Lyudmila Anatolievna Kazarinova, Moscow (RU); Sergey Viktorovich Gronskiy, Moscow (RU); Ekaterina Aleksandrovna Kutukova, Moscow (RU); Natalia Pavlovna Zakataeva, Moscow (RU)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO., INC., Tokyo (JP)

(21) Appl. No.: 09/988,350

(22) Filed: Nov. 19, 2001

(30) Foreign Application Priority Data
Nov. 22, 2000 (RU) 2000128988

(51) Int. Cl. C12P 19/40; C12N 1/21

(52) U.S. Cl. 435/88; 435/252.3

(57) ABSTRACT

Xanthosine-5′-monophosphate is produced by cultivating the bacterium which has a resistance to growth inhibition by an inhibitor selected from the group consisting of inhibitors of cell membrane biosynthesis and/or functioning, phosphorylation inhibitors, uncoupling agents, RNA-polymerase inhibitors and methionine analogs, and has an ability to produce xanthosine-5′-monophosphate according to produce and accumulate xanthosine-5′-monophosphate in the culture, and recovering the xanthosine-5′-monophosphate therefrom.

### TECHNICAL FIELD

The present invention relates to a fermentative method for the producing of xanthosine-5′-monophosphate, and microorganisms for use therein.

Xanthosine-5′-monophosphate (XMP) is an intermediate of purine nucleotide biosynthesis, and is used as an industrial raw material for the production of guanosine-5′-monophosphate(GMP), which has been known as a flavoring agent [Kuninaka (1960), J. Agr. Chem. Soc. Japan, 34, 489], and a starting material for synthesizing pharmaceuticals [U.S. Pat. No. 5,736,530].

### DESCRIPTION OF THE RELATED ART

Known methods for producing xanthosine-5′-monophosphate by direct fermentative processes include a method using various strains of Coryneform bacteria. Guanine-requiring or adenine-guanine requiring mutants of Corynebacterium glutamicum (Micrococcus glutamicus) and Brevibacteuium ammoniagenes (now renamed as Corynebacterium ammoniagenes) were found to accumulate a large amount of XMP under appropriate fermentation conditions [Misawa et al., 1964. Agr. Biol. Chem., 28, 690-693, Misawa et al., 1964. Agr. Biol., Chem., 28, 694-699, Demain et al., 1965. Appl. Vicrobiol., 13, 757-765; Misawa et al., 1969. Agr. Biol. Chem., 33, 370-376]. The accumulation of XMP by these strains seems to be due to the direct excretion of de novo synthesized nucleotide from the cells, because XMP pyrophosphorylase (xanthine phosphorybosyltransferase) was very low or deficient, and exogenously supplied xanthine was not converted to XMP by the growing cells [Misawa et al., 1964. Agr. Bio. Chem., 28, 694-699].

Besides, the accumulation of XMP together with IMP by adenine-requiring or adenine-guanine-requiring mutants of B. subtilis with weak 5′-nucleotidase activity was reported [Akiya et al., 1972. Agr. Biol. Chem., 36, 227-234].

A process using an adenine leaky, guanine requiring mutant of Corynebacterium ammoniagenes with weak nucleotidase activity and much higher productivity of XMP was described later [Fujio et al., 1984. J. Ferment. Technol., 62, 131].

Further, attempts to increase the productivity of Corynebacterium ammoniagenes xanthosine-5′-monophosphate producing strains by imparting additional properties to them also have been made. It has been found that the productivity of xanthosine-5′-monophosphate producing strains of Corynebacterium ammoniagenes can be greatly improved by endowing the adenine-guanine-requiring mutants with sensitivity to lysozyme [Korean Patent Publication No. 86-248 and No. 89-540] or resistance to cell wall inhibitory antibiotics [Japanese Patent Laid-Open No. 60-156399 A2].

Both the sensitivity to lysozyme and the resistance to the antibiotics are obviously involved in cytoplasmic membrane permeability for xanthosine-5′-monophosphate.

It is well known that a variety of treatments which put microbial cells under stress (temperature, irradiation, starvation, inhibitors and antibiotics) could lead to a breakdown of RNA and DNA with the subsequent excretion of nucleic acid derivatives [A. Demain (1968). Production of purine nucleotides by fermentation. In: Progress in Industrial Microbiology, Vol. 18. Ed. D. J. D. Hockenhull. J.&A. Churchill Ltd., London]. Nowadays it is generally accepted that the penetration of the metabolites across the cytoplasmic membrane is usually mediated by more or less specific efflux transporter proteins [Pao et al., 1998. Microbial. Mol. Biol. Rev., 62, 1-34; Paulsen et al., 1998. J. Mol. Biol., 277, 573-592; Saier et al., 1999. J. Mol. Microbiol. Biotechnol. (1999), 1, 257-279]. In turn, it is reasonable to suggest that these transporters may be induced or activated under the stress conditions. It was shown many years ago [Billen, D. (1957), Arch. Bichem. Biophys., 67, 333-340] that UV or X-rays irradiated Escherichia coli cells excrete free bases, ribosides, mononucleotides and ATP. Such liberation was not the result of cell lysis since no DNA derivatives or peptides were found. The requirements of glucose and inorganic phosphate for maximal release and the inhibition by arsenate or low temperature indicate that enzymatic action was involved, possibly including transporter protein synthesis.

In addition, a deficiency of manganese ions (Mn²⁺) in the culture of an adenine-leaky Corynebacterium ammoniagenes auxotroph caused “alteration in the membrane permeability barrier” for inosine-5′-monophosphate, resulting in prominent accumulation of the nucleotide [Furuya et al., 1970. Agr. Biol. Chem. 34, 210-217]. It was later established that the manganese ions deficiency affects the functioning of manganese-dependent ribonucleotide reductase, present in Coryneform bacteria, and induces unbalanced growth stress [Auling et al., 1980. Arch. Microbiol, 127, 105-114; Willing et al., 1988. Eur.J.Biochem., 170, 603-611]. Under this condition the cells display filamentous growth, excrete proteins and some metabolites into the culture medium.

An increased productivity of inosine-5′-monophosphate in the presence of excessive manganese ions by some Brevibacterium ammoniagenes mutants was suggested to be also caused by “improved permeability” for IMP excretion. These mutants exhibited an increased sensitivity to variety of antibiotics, detergents, dyes and lysozyme [Teshiba, S. and A. Furuya, 1983. Agr. Biol. Chem., 47, 1035-1041], obviously related to alterations of the bacterial membrane.

On the other hand, in Bacillus subtilis guanosine production was markedly improved by the introduction of the mutations which confer resistance to the inhibiting concentration of methionine and methionine analog, DL-methionine sulfoxide [Matsui et al., 1977. Appl. Environ. Microbiol., 34, 337-341; Matsui et al., 1979. Agr. Biol. Chem., 43, 1317-1323]. Methionine sulfoxide resistance mainly caused decrease of specific activity of 5′-nucleotidase and partial losses of inhibition and repression of IMP dehydrogenase by GMP [Matsui et al., 1977. Appl. Environ. Microbiol., 34, 337-341] Furthermore, repression and inhibition of PRPP amidotransferase by GMP were also lost [Matsui et al., 1979. Agr. Biol. Chem., 43, 1317-1323]. IMP dehydrogenase, which converts IMP to XMP, is the first enzyme of pathway specific for GMP synthesis, and is generally regulated by GMP [Nishikawa et al., 1967. J. Biochem., 62, 92]. PRPP amidotransferase is the first enzyme of purine nucleotide biosynthesis pathway, and is regulated by AMP and GMP [Nishikawa et al., 1967. J. Biochem., 62, 92; Sato and Shiio., 1970. J. Biochem., 68, 763]. However, the resistance to methionine analogs was never used for the improvement of XMP producing strains of Coryneform bacteria.

### DISCLOSURE OF THE INVENTION

The present invention has been taking the aforementioned viewpoints into consideration, an object of the present invention is to provide a more efficient method for the production of xanthosine-5′-monophosphate in high yield for industrial purpose and microorganisms which can be used in the method.

To this end, the present inventors after many studies on bacteria producing xanthosine-5′-monophosphate, found that microorganisms belonging to Corynebacterium ammoniagenes and having newly discovered mutation which confer resistance to the inhibitors of cell membrane biosynthesis and/or functioning, phosphorylation inhibitors, uncoupling agents, RNA-polymerase inhibitors and methionine analogs produce and accumulate a considerably more quantity of xanthosine-5′-monophosphate in a medium. The investigation of series of mutants showed the direct correlation between resistance to such compounds and accumulation of xanthosine-5′-monophosphate.

Heretofore, it was not recognized that the productivity of xanthosine-5′-monophosphate could be improved by endowing an xanthosine-5′-monophosphate producing microorganism with such traits.

Therefore work was continued on the basis of this finding to complete the present invention.

Thus, the present invention is as follows.

(1) Coryneform bacterium which has a resistance to growth inhibition by an inhibitor selected from the group consisting of inhibitors of cell membrane biosynthesis and/or functioning, phosphorylation inhibitors, uncoupling agents, RNA-polymerase inhibitors and methionine analogs, and has an ability to produce xanthosine-5′-monophosphate.

(2) Coryneform bacterium which has an ability to produce xanthosine-5′-monophosphate, and has a resistance to glycine.

(3) Coryneform bacterium which has an ability to produce xanthosine-5′-monophosphate, and has a resistance to polymyxin.

(4) Coryneform bacterium which has an ability to produce xanthosine-5′-monophosphate, and has a resistance to oligomycin.

(5) Coryneform bacterium which has an ability to produce xanthosine-5′-monophosphate, and has a resistance to carbonyl cyanide m-chlorophenylhydrasone (CCCP).

(6) Coryneform bacterium which has an ability to produce xanthosine-5′-monophosphate, and has a resistance to rifampicin.

(7) Coryneform bacterium which has an ability to produce xanthosine-5′-monophosphate, and has a resistance to methionine analog, wherein the methionine analog is selected from the group consisting of DL-methionine sulfoxide, L-methionine sulfoxide, DL-methionine sulfone and L-methionine sulfone.

(8) The coryneform bacterium according to any one of above (1) to (7), wherein the bacterium belongs to Corynebacterium ammoniagenes.

(9) The coryneform bacterium according to (2), wherein the bacterium is Corynebacterium ammoniagenes AGRI 10-52(VKPM B-8006).

(10) The coryneform bacterium according to (3), wherein the bacterium is Corynebacterium ammoniagenes AGRI 101-51(VKPM B-8010).

(11) The coryneform bacterium according to (4), wherein the bacterium is Corynebacterium ammoniagenes AGRI 67-52(VKPM B-8004).

(12) The coryneform bacterium according to (5), wherein the bacterium is Corynebacterium ammoniagenes AGRI 97-52(VKPM B-8008).

(13) The coryneform bacterium according to (6), wherein the bacterium is Corynebacterium ammoniagenes AGRI 93-38(VKPM B-8003).

(14) The coryneform bacterium according to (7), wherein the bacterium is Corynebacterium ammoniagenes AGRI 11-51(VKPM B-8005).

(15) The coryneform bacterium according to (7), wherein the bacterium is Corynebacterium ammoniagenes AGRI 47-51(VKPM B-8007).

(16) A method for producing xanthosine-5′-monophosphate by fermentation comprising the steps of cultivating the bacterium illustrated in any one of above (1) to (15) in a medium to produce and accumulate xanthosine-5′-monophosphate in the culture, and recovering the xanthosine-5′-monophosphate therefrom.

The present invention will be explained in detail below.

From the above findings of the inventors, they considered it is reasonable to presume that some mutation affecting cell membrane functioning, DNA replication, transcription or translation machinery may mimic stress conditions and induce an enhancement of a specific transporter activity thus increasing nucleic acid derivative, and more specifically, xanthosine-5′-monophosphate accumulation. Furthermore, in view of the fact, that the transporter mediated excretion may depend on the energy state of the bacterial cell, the mutations enhancing ATP-regeneration activity may also be useful for xanthosine-5′-monophosphate producing strain improvement.

The microorganisms of the present invention may be obtained from microorganisms inherently having an ability to produce xanthosine-5′-monophosphate by imparting thereto the specified resistance. Alternatively, the microorganisms of the present invention may be also obtained by imparting an ability to produce xanthosine-5′-monophosphate to microorganisms having the specified resistance.

The term “bacterium which has a resistance to inhibitors of cell membrane biosynthesis and/or functioning” means a microorganism derived from strain of bacterium belonging to Coryneform bacteria as the parent strain and modified in genetic properties so that it can grow in medium containing inhibitors of cell membrane biosynthesis and/or functioning. The term “inhibitor of cell membrane biosynthesis and/or functioning” means a compound (e.g. glycine, polymyxin, gramicidin) which inhibits cytoplasmic membrane biosynthesis or affects its normal functioning. Thus, as used herein the term “a resistance to growth inhibition by inhibitors of cell membrane biosynthesis and/or functioning” means that the mutant is capable of growing in nutrient medium containing the compound as an inhibitor in an amount which would inhibit the growth of the parent strains.

For example, a microorganism which can form colonies within 3-5 days by cultivation with 32° C. on an agar plate containing 40 g/L or more, preferably 50 g/L or more of glycine, 40 mg/L or more, preferably 50 mg/L or more of polymyxin, 5 mg/L or more, preferably 10 mg/L or more of gramicidin is resistant to these drugs.

The term “bacterium which has a resistance to phosphorylation inhibitor” means a microorganism derived from strain of bacterium belonging to Coryneform bacteria as the parent strain and modified in genetic properties so that it can grow in medium containing phosphorylation inhibitor. The term “phosphorylation inhibitor” means a compound (e.g. oligomycin) which inhibits ATP synthesis from ADP and Pᵢ by F₀/F₁ ATPase (ATP synthase). Thus, as used herein the term “a resistance to growth inhibition by phosphorylation inhibitors” means that the mutant is capable of growing in nutrient medium containing the compound as an inhibitor in an amount which would inhibit the growth of the parent strains.

For example, a microorganism which can form colonies within 3 days by cultivation with 32° C. on an agar plate containing 50 mg/L or more, preferably 100 mg/L or more of oligomycin is resistant to oligomycin.

The term “bacterium which has a resistance to uncoupling agents” means a microorganism derived from strain of bacterium belonging to Coryneform bacteria as the parent strain and modified in genetic properties so that it can grow in medium containing uncoupling agent. The term “uncoupling agent” means a compound [e.g. dinitrophenol, carbonyl cyanide m-chlorophenyl hydrazone (CCCP), p-trifluoromethoxy carbonyl cyanide phenyl hydrazone (FCCP)] which abolish the obligatory linkage between the respiratory chain and the phosphorylation system. Thus, as used herein the term “a resistance to uncoupling agent” means that the mutant is capable of growing in nutrient medium containing the compound as an inhibitor in an amount which would inhibit the growth of the parent strains.

For example, a microorganism which can form colonies within 3 days by cultivation with 32° C. on an agar plate containing 2 mg/L or more, preferably 4 mg/L or more of CCCP or FCCP is resistant to CCCP or FCCP.

The term “bacterium which has a resistance to RNA-polymerase inhibitor” means a microorganism derived from strain of bacterium belonging to Coryneform bacteria as the parent strain and modified in genetic properties so that it can grow in medium containing RNA-polymerase inhibitor. The term “RNA-polymerase inhibitor” means a compound (e.g. rifampicin (also called as rifampin)) which inhibits the activity of the DNA-dependent RNA polymerase. Thus, as used herein the term “a resistance to RNA-polymerase inhibitor” means that the mutant is capable of growing in nutrient medium containing the compound as an inhibitor in an amount which would inhibit the growth of the parent strains.

For example, a microorganism which can form colonies within 3 days by cultivation with 32° C. on an agar plate containing 5 mg/L or more, preferably 15 mg/L or more of rifampicin is resistant to rifampicin.

The term “bacterium which has a resistance to methionine analogue” means a microorganism derived from strain of bacterium belonging to Coryneform bacteria as the parent strain and modified in genetic properties so that it can grow in medium containing methionine analogue. The term “methionine analogue” means a compound similar in structure to methionine [DL-methionine sulfoxide, L-methionine sulfoxide, DL-methionine sulfone and L-methionine sulfone]. Thus, as used herein the term “a resistance to methionine analogue” means that the mutant is capable of growing in nutrient medium containing the compound as an inhibitor in an amount which would inhibit the growth of the parent strains.

For example, a microorganism which can form colonies within 5 days by cultivation with 32° C. on an agar plate containing 5 g/L or more, preferably 10 g/L or more of DL-methionine sulfoxide or L-methionine sulfoxide, or 5 g/L or more, preferably 10 g/L or more of DL-methionine sulfone or L-methionine sulfone is resistant to these drugs.

The “coryneform bacteria” referred to in the present invention include bacteria having been hitherto classified into the genus Brevibacterium but united into the genus Corynebacterium at present [Int. J. Syst. Bacteriol., 41, 255 (1981)], and include bacteria belonging to the genus Brevibacterium closely relative to the genus Corynebacterium. Examples of such coryneform bacteria include the followings.

Corynebacterium ammoniagenes (Brevibacterium ammoniagenes)

Corynebacterium acetoacidophilum

Corynebacterium acetoglutamicum

Corynebacterium alkanolyticum

Corynebacterium callunae

Corynebacterium glutamicum

Corynebacterium lilium (Corynebacterium glutamicum)

Corynebacterium melassecola

Corynebacterium thermoaminogenes

Corynebacterium herculis

Brevibacterium divaricatum (Corynebacterium glutamicum)

Brevibacterium flavum (Corynebacterium glutamricum)

Brevibacterium immariophilum

Brevibacterium lactofermentum (Corynebacterium glutamicum)

Brevibacterium roseum

Brevibacterium saccharolyticum

Brevibacterium thiogenitalis

Brevibacterium album

Brevibacterium cerinum

Microbacterium ammoniaphilum

Specifically, the following strains of these bacteria are exemplified:

Corynebacterium ammoniagenes (Brevibacterium ammoniagenes) ATCC6871, ATCC6872, VKPM B-6307

Corynebacterium acetoacidophilum ATCC13870

Corynebacterium acetoglutamicum ATCC15806

Corynebacterium alkanolyticum ATCC21511

Corynebacterium callunae ATCC15991

Corynebacterium glutamricum ATCC13020, ATCC13032, ATCC13060

Corynebacterium lilium (Corynebacterium glutamicum) ATCC15990

Corynebacterium melassecola ATCC17965

Corynebacterium thermoaminogenesAJ12340 (FERM BP-1539)

Corynebacterium herculis ATCC13868

Brevibacterium divaricatum (Corynebacterium glutamicum) ATCC14020

Brevibacterium flavum (Corynebacterium glutamicum) ATCC13826, ATCC14067

Brevibacterium immariophilum ATCC14068

Brevibacterium lactofermentum (Corynebacterium glutamicum) ATCC13665, ATCC13869

Brevibacterium roseum ATCC13825

Brevibacterium saccharolyticum ATCC14066

Brevibacterium thiogenitalis ATCC19240

Brevibacterium album ATCC15111

Brevibacterium cerinum ATCC15112

Microbacterium ammoniaphilum ATCC15354

In addition to the properties already mentioned they may have other specific properties such as various nutrient requirement, drug resistance, drug sensitivity and drug dependence without departing from the scope of the present invention. Further the microorganisms of the present invention may be modified by genetic recombination techniques to increase the activity of the enzyme involved in xanthosine-5′-monophosphate biosynthesis.

The mutant microorganisms useful in carrying out the present invention can be obtained by mutation using conventional mutagenesis such as ultraviolet ray irradiation, X-ray irradiation, radioactive ray irradiation, and a treatment with chemical mutagens followed by the selection by the replica method. The preferred mutagen is N-nitro-N′-methyl-N-nitrosoguanidine (hereafter referred to as NTG).

Thus, it is possible to subject any known strain belonging to coryneform bacteria such as Corynebacterium ammoniagenes inherently having an ability to produce xanthosine-5′-monophosphate to one of the above mutation procedure for obtaining a mutant strain, and then to test the mutant strain to determine whether it satisfies the above-requirement of the present invention concerning resistance to growth inhibition by inhibitors of cell membrane biosynthesis and/or functioning, phosphorylation inhibitors, uncoupling agents, RNA-polymerase inhibitors or methionine analogues, and it is therefore suitable for use in the invention. Strains mutated as mentioned above are screened by culturing in a nutrient medium and selecting a strain having the ability to produce xanthosine-5′-monophosphate in greater yields than its parent strain and the obtained strains are used in this invention.

Strains satisfying the requirement of the present invention may be obtained also by genetic recombination techniques which are well-known to the person skilled in the art.

The above-mentioned features of resistance may be combined in one strain by sequential selection or genetic recombination techniques.

Representative examples of the strain to be used in the practice of the invention are AGRI101-51 (VKPM B-8010), AGRI10-52 (VKPM B-8006), AGRI67-52 (VKPM B-8004), AGRI97-52 (VKPM B-8008), AGRI11-51(VKPM B-8005), AGRI47-51(VKPM B-8007), and AGRI93-38(VKPM B-8003). The Coryneform bacteria to be used in producing xanthosine-5′-monophosphate in accordance with the invention have the same bacteriological properties as the parent strain except for the resistance to growth inhibition by inhibitors of cell membrane biosynthesis and/or functioning, phosphorylation inhibitors, uncoupling agents, RNA-polymerase inhibitors and methionine analogues, and ability to produce at higher yields of xanthosine-5′-monophosphate. The deposition numbers

(VKPM B-Numbers) are those for the microorganisms which have been deposited in the Russian National Collection of Industrial Microorganisms (VKPM), 1-st Dorozhny Proezd, b.1, Moscow, Russia. VKPM B-8003 to 8010 were deposited in the above depositary authority on Jul. 17, 2000, and transferred from the original deposit to international deposit based on Budapest Treaty on Oct. 15, 2001.

These strains have been derived from Corynebacterium ammoniagenes AGRI45-11 (VKPM B-8009) as the parent strain, which is a streptomycin-resistant derivative of the xanthosine-5′-monophosphate-producing strain Corynebacterium ammoniagenes AJ13606. The strain Corynebacterium ammoniagenes AJ13606, in turn, has been obtained from inosine-5′-monophosphate-producing strain Corynebacterium ammoniagenes AG98-79, a derivative of the known strain Corynebacterium ammoniagenes 225-5 (VKPM B-1073) [SU Patent 515783] by sequential introducing into genome of the strain AG98-79 a series of mutations: guanine-leaky requirement, sensitivity to high temperature, and resistance to sulfaguanidine.

The xanthosine-5′-monophosphate producing microorganisms obtained in the above manner may be cultivated in the same manner as the conventional cultivation microorganisms. Thus, as the medium, there may be used a liquid culture medium containing a carbon source or sources, a nitrogen source or sources and metal ions and, as necessary, other nutrients such as amino acids, nucleic acids and vitamins, as the carbon source, for instance, there may be used glucose, fructose, ribose, maltose, mannose, sucrose, starch, starch hydrolyzate, molasses and so forth. As the nitrogen source, there may be used organic nitrogen sources such as pepton, corn steep liquor, soybean meal, yeast extract and urea, and, further, inorganic nitrogen sources such as ammonium salts of sulfuric, nitric, hydrochloric, carbonic and other acids, gaseous ammonia and aqueous ammonia, either singly or combination. As other nutrients, inorganic salts, amino acids, vitamins and so forth necessary for the bacterial growth are appropriately selected and used either singly or in combination.

The cultivation is generally carried out under aerobic conditions. The medium preferably has a pH within the range of 5 to 9. The cultivation temperature is generally selected within the range of 25° C. to 40° C. so that it may be appropriate for the growth of the microorganisms used and for the accumulation of xanthosine-5′-monophosphate. The cultivation is preferably conducted until the accumulation of xanthosine-5′-monophosphate becomes substantially maximal. Generally, 1 to 6 days of cultivation achieves this end.

For the separation and recovery of xanthosine-5′-monophosphate from the resultant culture broth, there may be used per se known usual techniques of purification.

The method of production of xanthosine-5′-monophosphate according to the present invention is advantageous from the industrial point of view in that it causes accumulation of xanthosine-5′-monophosphate in larger amounts with little by-production of inosine-5′-monophosphate, xanthosine or xanthine.

### BEST MODE FOR CARRYING OUT THE INVENTION

The following examples are intended to illustrate this invention more concretely.

### EXAMPLE 1

### Selection of the Mutants Resistant to Glycine

Glycine in concentrations between 1 and 6% affects cell wall synthesis by inhibiting D-alanyl:D-alanyl ligase and alanine racemase. In Escherichia coli the mutants with an increased tolerance to glycine exhibit also increased sensitivity to penicillin G (Wijsman and Pafort, 1974. Mol.Gen.Genet., 128, 349-357). Thus, by selecting glycine-resistant mutants it is possible to obtain strains with distorted function in cell envelope. This defect may mimic stress condition and induce a transporter involved in XMP excretion. Therefore, the mutants resistant to growth inhibition by glycine were selected.

Corynebacterium ammoniagenes AGRI45-11 (VKPM B-8009) was treated with 50 μg/ml of NTG for 20 minutes. Then cells were plated on PYM medium having the composition (g/L): peptone—10.0, yeast extract—10.0, meat extract—5.0, NaCl—2.5, agar—20.0, and containing 40 g/L, or 50 g/L, or 60 g/L glycine. The inoculated plates were incubated at 30° C. for 5 days. From among the colonies that appeared, the most productive strain Corynebacterium ammoniagenes AGRI10-52 (VKPM B-8006) was selected.

This strain and the parent strain Corynebacterium ammoniagenes AGRI45-11 (VKPM B-8009) were each cultivated at 32° C. for 20 h with aeration in a seed medium having the composition (g/L): glucose—20.0, peptone—10.0, yeast extract 10.0, NaCl—2.5, pH 7.2 contained in 20×200 mm test tubes. Then 0.3 ml of the obtained culture was inoculated into 3 ml of the fermentation medium having the following composition (see below), in 20×200 mm test tubes, and cultivated at 32° C. for 72 hours with a rotary shaker.

After the cultivation, an accumulated amount of XMP in the medium was determined by known method.

The results are presented in Table 1. As shown in Table 1, the AGRI10-52 strain resistant to glycine accumulated more XMP than the parental strain.

Composition of the minimal medium, g/L

| | |
|---|---|
| Glucose | 90.0 |
| Urea | 7.2 |
| Glutamate, monosodium salt | 20.0 |
| Mamenou(T-N) | 1.4 |
| KH₂PO₄ | 15.0 |
| K₂HPO₄ | 15.0 |
| MgSO₄.7H₂O | 10.0 |
| CaCl₂.2H₂O | 0.1 |
| MnCl₂.4H₂O | 0.01 |
| ZnSO₄.7H₂O | 0.001 |
| FeSO₄.7H₂O | 0.01 |
| Biotin | 0.00003 |
| Ca.pantotenate | 0.01 |
| Thiamine-HCl | 0.005 |
| Adenine | 0.025 |
| Guanine | 0.025 |
| pH (adjusted with KOH) | 7.2 |

TABLE 1

| | Growth in the | |
|---|---|---|
| | presence of 6% | XMP.2Na.7H₂O |
| Strain | glycine | g/L |
| AGRI45-11 | − | 23.5 |
| AGRI10-52 | + | 28.0 |

Note.
+: growth;
−: no growth

### EXAMPLE 2

### Selection of the Mutants Resistant to Polymyxin B

Polymyxin B is a polypeptide antibiotics effective against Gram-negative bacteria. The cell membrane of the bacteria is considered to be the target of the antibiotic. The present inventors found that polymyxin B at high concentrations (40-50 μg/ml) inhibited the growth of the Gram-positive bacterium Corynebacterium ammoniagenes. The mutations conferring resistance to polymyxin B may affect cytoplasmic membrane and mimic a stress condition, inducing XMP efflux transporter. Therefore the mutants resistant to growth inhibition by the drug were selected.

Corynebacterium ammoniagenes AGRI45-11 (VKPM B-8009) was treated with NTG, and cells were plated on the PYM medium as in Example 1, containing 45 μg/ml, or 50 μg/ml, or 55 μg/ml, or 60 μg/ml polymyxin. The inoculated plates were incubated at 30° C. for 5 days. From among the colonies that appeared the most productive strain Corynebacterium ammoniagenes AGRI101-51 (VKPM B-8010) was selected.

This strain and the parent strain Corynebacterium ammoniagenes AGRI45-11 (VKPM B-8009) were each cultivated at 32° C. for 20 h with aeration in a seed medium as in the Example 1. Then 0.3 ml of the obtained culture was inoculated into 3 ml of the fermentation medium of the Example 1, in 20×200 mm test tubes, and cultivated at 32° C. for 72 hours with a rotary shaker. After the cultivation, an accumulated amount of XMP in the medium was determined by known method.

The results are presented in Table 2. As shown in Table , the AGRI101-51 strain resistant to polymyxin accumulated more XMP than the parental strain.

TABLE 2

| | Growth in the | |
|---|---|---|
| | presence of 45 μg/ml | XMP.2Na.7H₂O |
| Strain | polymyxin | g/L |
| AGRI45-11 | − | 23.5 |
| AGRI 101-51 | + | 27.6 |

See the note to the Table 1.

### EXAMPLE 3

### Selection of the Mutants Resistant to Rifampicin

Rifampicin and its derivatives are antibiotics that inhibit the activity of the DNA-dependent RNA polymerase by binding to the β-subunit of the enzyme and preventing the initiation of transcription [Hartman et al., 1967. Biochim. Biophys. Acta, 145, 843-844; Linn et al., 1975. J. Bacteriol., 122, 1387-1390]. Mutations to rifampicin resistance have been reported to have pleiotropic effects on complex metabolic processes of different bacteria [Kane et al., 1979. J. Bacteriol., 137, 1028-1030; Jin and Gross, 1989. J. Bacteriol., 171, 5229-5231; Livshits and Sukhodolets, 1973. Genetika, 9, 102-111] resembling a response to stress condition. Therefore, the mutants resistant to growth inhibition by rifampicin were selected and tested for their XMP productivity.

The parental strain Corynebacterium ammoniagenes AGRI45-11 (VKPM B-8009) was streaked onto PYM medium of Example 1, containing 5, 15, 30 or 50 μg/ml of rifampicin instead of glycine and inoculated plates were incubated at 34° C. for 3 days. The mutants resistant to rifampicin was selected from among spontaneously appeared colonies. Among the mutants the most productive strain Corynebacterium ammoniagenes AGRI93-38 (VKPM B-8003) was selected.

This strain and the parent strain Corynebacterium ammoniagenes AGRI45-11 (VKPM B-8009) were each cultivated at 32° C. for 20 h with aeration in a seed medium as in the Example 1. Then 0.3 ml of the obtained culture was inoculated into 3 ml of the fermentation medium of the Example 1, in 20×200 mm test tubes, and cultivated at 32° C. for 72 hours with a rotary shaker. After the cultivation, an accumulated amount of XMP in the medium was determined by known method

The results are presented in Table 3. As shown in Table 3, the AGRI93-38 strain resistant to rifampicin accumulated about 10% more XMP than the parental strain.

TABLE 3

| | Growth with 15 μg/ml | XMP.2Na.7H₂O |
|---|---|---|
| Strain | rifampicin | g/L |
| AGRI45-11 | − | 23.5 |
| AGRI93-38 | + | 26.0 |

See the note to the Table 1.

### EXAMPLE 4

### Selection of the Mutants Resistant to Oligomycin

Oligomycin is a well known phosphorylation inhibitor which blocks ATP synthesis by the F₀/F₁ ATPase. The mutations overcoming the effect of the antibiotic may have increased ATP-generating activity. In turn, the activation of ATP production may have positive effect on transporter mediated purine nucleotide excretion.

A series of mutants resistant to 50 or 100 μg/ml oligomycin was selected from the strain Corynebacterium anmoniagenes AGRI45-11 (VKPM B-8009) using the procedure, described in the Example 1, but oligomycin was used instead of glycine. Some of them proved to be more productive than the parental strain. The best mutant, strain Corynebacterium ammoniagenes AGRI67-52 (VKPM B-8004) when tested in parallel as in Example 1 accumulated about 22 % more XMP than the parental strain Corynebacterium ammoniagenes AGRI45-11 (VKPM B-8009) (Table 4 ).

TABLE 4

| | Growth in the presence | XMP.2Na.7H₂O |
|---|---|---|
| Strain | of 100 μg/ml oligomycin | G/L |
| AGRI45-11 | − | 23.5 |
| AGRI67-52 | + | 28.5 |

See the note to the Table 1.

### EXAMPLE 5

### Selection of the Mutants Resistant to Carbonyl Cyanide m-chlorophenyl Hydrazone

Carbonyl cyanide m-chlorophenyl hydrazone (CCCP) is well known uncoupling agent which abolish the obligatory linkage between the respiratory chain and the phosphorylation system. The mutations overcoming the effect of the drug may enhance energy metabolism of cell and increase ATP-generating activity. In turn, the activation of ATP production may have positive effect on transporter mediated purine nucleotide excretion.

Corynebacterium ammoniagenes AGRI45-11 (VKPM B-8009) was treated with NTG, and cells were plated on PYM medium as in Example 1, containing 2, 4 or 6 μg/ml of CCCP instead of glycine. The inoculated plates were incubated at 30° C. for 5 days. From among the colonies that appeared the most productive strain Corynebacterium ammoniagenes AGRI97-52 (VKPM B-8008) was selected. This strain and the parent strain Corynebacterium ammoniagenes AGRI45-11 (VKPM B-8009) were each cultivated at 32° C. for 20 h with aeration in a seed medium as in the Example 1. Then 0.3 ml of the obtained culture was inoculated into 3 ml of the fermentation medium of the Example 1, in 20×200 mm test tubes, and cultivated at 32° C. for 72 hours with a rotary shaker. After the cultivation, an accumulated amount of XMP in the medium was determined by known method.

The results are presented in Table 5. As shown in Table5, the strain AGRI97-52 accumulated about 15% more XMP than the parental strain.

TABLE 5

| | Growth with 4 | XMP.2Na.7H₂O |
|---|---|---|
| Strain | μg/ml CCCP | g/L |
| AGRI45-11 | − | 23.1 |
| AGRI97-52 | + | 26.6 |

See the note to the Table 1.

### EXAMPLE 6

### Selection of the Mutants Resistant to Methionine Sulfoxide

DL-methionine and its analogue, the glutamine antagonist, DL-methionine sulfoxide in high concentration inhibited the growth of the XMP producer Corynebacterium ammoniagenes AGRI45-11 (VKPM B-8009) strain. It is known that methionine sulfoxide-resistant mutants of Bacillus subtilis may have improved guanosine production (Matsui et al., Appl. Environ. Microbiol., 34, 337-341, 1977; Matsui et al., Agric. Biol. Chem., 43 (6), 1317-1323, 1979; Matsui et al., Agric. Biol. Chem., 46(9), 2347-2352, 1982). Therefore, the mutants resistant to the analogue were obtained.

Corynebacterium ammoniagenes AGRI45-11 (VKPM B-8009) was treated with NTG as in Example 1, aliquots were introduced into 20×200 mm test tubes and incubated at 32° C. for 20 h with shaking in minimal medium having the following composition (see below) with a rotary shaker. Then the cells were collected and plated on the minimal medium agar and containing several combinations of the inhibitors:

1. DL-methionine sulfoxide (5 mg/ml)+DL-methionine (7.5 mg/ml);

2. DL-methionine sulfoxide (10 mg/ml)+DL-methionine (7.5 mg/ml);

3. DL-methionine sulfoxide (5 mg/ml)+DL-methionine (20 mg/ml);

4. DL-methionine sulfoxide (10 mg/ml)+DL-methionine (20 mg/ml);

Composition of the minimal medium, g/L

| | |
|---|---|
| Glucose | 20.0 |
| Urea | 2.0 |
| KH₂PO₄ | 1.0 |
| K₂HPO₄ | 3.0 |
| MgSO₄.7H₂O | 0.3 |
| CaCl₂.2H₂O | 0.1 |
| MnCl₂.4H₂O | 0.0036 |
| ZnSO₄.7H₂O | 0.001 |
| FeSO₄.7H₂O | 0.01 |
| Biotin | 0.00003 |
| Ca.pantotenate | 0.01 |
| Thiamine-HCl | 0.005 |
| Adenine | 0.025 |
| Guanine | 0.025 |
| pH (adjusted with KOH) | 7.2 |
| Agar (in solid medium) | 20.0 |

Mutants which appeared after 4 to 8 days incubation were picked up and tested for their XMP productivity. Among them the strain Corynebacterium ammoniagenes AGRI11-51 (VKPM B-8005) was selected. This strain and the parent strain Corynebacterium anmoniagenes AGRI45-11 (VKPM B-8009) were each cultivated at 32° C. for 20 h with aeration in a seed medium as in the Example 1. Then 0.3 ml of the obtained culture was inoculated into 3 ml of the fermentation medium of the Example 1, in 20×200 mm test tubes, and cultivated at 32° C. for 72 hours with a rotary shaker. After the cultivation, an accumulated amount of XMP in the medium was determined by known method. The results are presented in Table 6. As shown in Table 6, the strain AGRI11-51 accumulated about 31 % more XMP than the parental strain.

TABLE 6

| | Growth with 10 mg/ml DL- | |
|---|---|---|
| | methionine sulfoxide + 20 | XMP.2Na.7H₂O |
| Strain | mg/ml DL-methionine | g/L |
| AGRI45-11 | − | 23.5 |
| AGRI11-51 | + | 31.1 |

See the note to the Table 1.

### EXAMPLE 7

### Selection of the Mutants Resistant to Methionine Sulfone

Another methionine analogue, DL-methionine sulfone in high concentration also inhibited the growth of the XMP producer Corynebacterium ammoniagenesAGRI45-11 (VKPM B-8009) strain. The mutants resistant to the analogue were obtained using the procedure described in Example 6, except for the only combinations of the inhibitors was applied: DL-methionine sulfone-10 mg/ml +DL-methionine-20 mg/ml.

Among the mutants which appeared after 4 to 8 days incubation the strain Corynebacterium ammoniagenes AGRI47-51 (VKPM B-8007) was selected. This strain and the parent strain Corynebacterium ammoniagenes AGRI45-11 (VKPM B-8009) were each cultivated at 32° C. for 20 h with aeration in a seed medium as in the Example 1. Then 0.3 ml of the obtained culture was inoculated into 3 ml of the fermentation medium of the Example 1, in 20×200 mm test tubes, and cultivated at 32° C. for 72 hours with a rotary shaker. The results are presented in Table 7.

The results are presented in Table 7.

TABLE 7

| | Growth with 10 mg/ml DL- | |
|---|---|---|
| | methionine sulfone + 20 mg/ml | XMP.2Na.7H₂O |
| Strain | DL-methionine | g/L |
| AGRI45-11 | − | 23.5 |
| AGRI47-51 | + | 32.2 |

See the note to the Table 1.

As shown in Table 7, the AGRI47-51 strain accumulated about 36% more XMP than the parental strain.

What is claimed is:

1. Coryneform bacterium which has a resistance to growth inhibition by an inhibitor selected from the group consisting of inhibitors of cell membrane biosynthesis and/or functioning, phosphorylation inhibitors, uncoupling agents, RNA-polymerase inhibitors and methionine analogs, and has an ability to produce xanthosine-5′-monophosphate.

2. Coryneform bacterium which has an ability to produce xanthosine-5′-monophosphate, and has a resistance to glycine.

3. Coryneform bacterium which has an ability to produce xanthosine-5′-monophosphate, and has a resistance to polymyxin.

4. Coryneform bacterium which has an ability to produce xanthosine-5′-monophosphate, and has a resistance to oligomycin.

5. Coryneform bacterium which has an ability to produce xanthosine-5′-monophosphate, and has a resistance to carbonyl cyanide m-chlorophenylhydrasone (CCCP).

6. Coryneform bacterium which has an ability to produce xanthosine-5′-monophosphate, and has a resistance to rifampicin.

7. Coryneform bacterium which has an ability to produce xanthosine-5′-monophosphate, and has a resistance to methionine analog, wherein the methionine analog is selected from the group consisting of DL-methionine sulfoxide, L-methionine sulfoxide, DL-methionine sulfone and L-methionine sulfone.

8. Coryneform bacterium according to any one of claims 1 to 7, wherein the bacterium belongs to Corynebacterium ammoniagenes.

9. The coryneform bacterium according to claim 2, wherein the bacterium is Corynebacterium ammoniagenes AGRI 10-52 (VKPM B-8006).

10. The coryneform bacterium according to claim 3, wherein the bacterium is Corynebacterium ammoniagenes AGRI 101-51 (VKPM B-8010).

11. The coryneform bacterium according to claim 4, wherein the bacterium is Corynebacterium ammoniagenes AGRI 67-52 (VKPM B-8004).

12. The coryneform bacterium according to claim 5, wherein the bacterium is Corynebacterium ammoniagenes AGRI 97-52 (VKPM B-8008).

13. The coryneform bacterium according to claim 6, wherein the bacterium is Corynebacterium ammoniagenes AGRI 93-38 (VKPM B-8003).

14. The coryneform bacterium according to claim 7, wherein the bacterium is Corynebacterium ammoniagenes AGRI 11-51 (VKPM B-8005).

15. The coryneform bacterium according to claim 7, wherein the bacterium is Corynebacterium ammoniagenes AGRI 47-51 (VKPM B-8007).

16. A method for producing xanthosine-5′-monophosphate by fermentation comprising the steps of cultivating the bacterium according to any one of claims 1 to 15 in a medium to produce and accumulate xanthosine-5′-monophosphate in the culture, and recovering the xanthosine-5′-monophosphate therefrom.
